(19) United States
(12) Patent Application Publication
(10) Pub. No.: US 2009/0123509 A1
(43) Pub. Date: May 14, 2009

(54) Biodegradable Colloidal Gels as Moldable Tissue Engineering Scaffolds

(76) Inventors: Cory Berkland, Lawrence, KS (US); Qun Wang, Lawrence, KS (US)

Correspondence Address: Workman Nydegger;1000 Eagle Gate Tower, 60 East South Temple, Salt Lake City, UT, 84111, US

(21) Appl. No.: 12/265,634

(22) Filed: Nov. 5, 2008

Related U.S. Application Data
(60) Provisional application No. 60/986,555, filed on Nov. 8, 2007.

(51) Int. Cl. A61K 9/14; A61K 35/12; A61K 9/00; A61P 43/00

(52) U.S. Cl. 424/422; 424/486; 424/93.7

(57) ABSTRACT

A colloid gel can include a plurality of positive charged particles mixed and associated with a plurality of negative charged particles so as to form a three-dimensional matrix having a plurality of pores defined by and disposed between the particles. The three-dimensional matrix can have shear thinning under shear and structure stability in the absence of shear. A method of manufacturing the colloid gel can include combining the positive charged particles with the negative charged particles, in a mold or in situ, so as to form the three-dimensional matrix having the plurality of pores.

### CROSS-REFERENCE TO RELATED APPLICATIONS

This patent application claims benefit of U.S. patent application Ser. No. 60/986,555, filed Nov. 8, 2007, which provisional application is incorporated herein by specific reference in its entirety.

### BACKGROUND

Tissue engineering is a multidisciplinary field that involves the development of biological substitutes that restore, maintain or improve tissue functions. This field has the potential of overcoming the limitations of conventional treatments by producing a supply of organ and tissue substitutes biologically tailored to a patient. There is a continuing need in biomedical sciences for scaffolds of biocompatible compositions which closely mimic the composition and structure of natural substrates and which can be used in manufacturing devices for implantation within or upon the body of an organism.

Several techniques have been developed to produce tissue engineering scaffolds from biodegradable and bioresorbable polymers. For synthetic polymers, these are usually based on solvent casting-particulate leaching, phase separation, gas foaming and fiber meshes. For natural collagen scaffolds, these can be made by freezing a dispersion/solution of collagen and then freeze-drying it. Freezing the dispersion/solution results in the production of ice crystals that grow and force the collagen into the interstitial spaces, thus aggregating the collagen. The ice crystals are removed by freeze-drying which involves inducing the sublimation of the ice and this gives rise to pore formation; therefore the water passes from a solid phase directly to a gaseous phase and eliminates any surface tension forces that can collapse the delicate porous structure. A major challenge for tissue engineering is to generate scaffolds which are sufficiently complex in mimicking the functions of natural substrates and yet not immunogenic. While tissue engineering scaffolds have been produced that can grow cells, an optional scaffold has not yet been obtained. Thus, research continues to search for improvements in tissue engineering scaffolds.

### SUMMARY

In one embodiment, a colloid gel for use as a tissue engineering scaffold can include: a plurality of positive charged particles; and a plurality of negative charged particles associated with the plurality of positive charged particles so as to form a three-dimensional matrix having a plurality of pores defined by and disposed between the particles, said three-dimensional matrix having shear thinning under shear and structure stability in the absence of shear. The particles can be biocompatible so as to be capable of being implanted into a subject or applied to a wound. The colloid gel can be used for a prosthesis, such as an endoprosthesis and/or an exoprosthesis.

In one embodiment, the colloid gel can be prepared by substituting only one of the particles with a polymer. The polymer can have various molecular weights; however, larger and/or longer polymers can be useful and more particle like. The polymer can be branched, crosslinked, or linear. The polymer can be substituted for either a positive particle or a negative particle, and a particle of opposite charge of the polymer can be combined therewith in order to prepare a colloid gel having the properties described herein for use as a tissue engineering scaffold.

In one embodiment, at least a portion of the plurality of positive charged particles and plurality of negatively charged particles are nanoparticles. Optionally, a majority of the plurality of positive charged particles and plurality of negatively charged particles are nanoparticles. For example, the plurality of positive charged particles and plurality of negatively charged particles can have a nano size, submicron size, and micron sizes.

In one embodiment, the colloid is disposed in a container, syringe, a catheter, an injection apparatus, or even within a subject.

In one embodiment, the colloid gel can include at least one bioactive agent disposed within the three-dimensional matrix. Optionally, the bioactive agent can be disposed within at least one particle. The negative charged particle can have one bioactive agent and the positive charged particle can have another bioactive agent. Also, the bioactive agent can be disposed within an interstitial space between the particles. Moreover, cells can be disposed and growing within the pores.

In one embodiment, a method for manufacturing a colloid gel can include: providing a plurality of positive charged particles; providing a plurality of negative charged particles; combining the positive charged particles with the negative charged particles so as to form a three-dimensional matrix having a plurality of pores defined by and disposed between the positive and negative charged particles. The three-dimensional matrix having shear thinning under shear and structure stability in the absence of shear. The shape of the matrix can be prepared in a mold or after being deposited within the body of a subject.

In one embodiment, the method can further include preparing a majority of the plurality of positive charged particles and plurality of negatively charged particles as nanoparticles. The nanoparticles can have a size as described herein.

In one embodiment, the method can further include introducing the colloid gel into a syringe. The syringe can then be used for introducing the colloid gel into a subject as an implant. Also, the colloid gel can be introduced into a medical device, such as a catheter, that is capable of introducing the colloid gel into a subject with shear thinning.

In one embodiment, the method further includes introducing at least one bioactive agent into the three-dimensional matrix. This can include introducing the bioactive agent into at least one particle. Also, this can include introducing the bioactive agent into an interstitial space between the particles.

In one embodiment, the method can include introducing cells into the pores. The cells can be introduced into the pores before, during, or after placement into a subject.

In one embodiment, a method for forming an implant in situ can include: providing a colloid gel formed by combining positive charged particles with negative charged particles so as to form a three-dimensional matrix having a plurality of pores defined by and disposed between the positive and negative charged particles, said three-dimensional matrix having shear thinning under shear and structure stability in the absence of shear; and injecting the colloid gel into a subject so as to form an implant.

In one embodiment, the method can further include preparing a majority of the plurality of positive charged particles and plurality of negatively charged particles as nanoparticles, and combining the positive charged particles and plurality of negatively charged particles to form the colloid gel. The method can further include introducing the colloid gel into a medical device, such as a catheter, pump, syringe, or the like that can implant the colloid gel into a subject. The method can also include shaping the colloid gel into a shape of the implant while within the subject.

These and other embodiments and features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only typical embodiments of the invention and are therefore not to be considered limiting of its scope. The invention will be described and explained with additional specificity and detail through the use of the accompanying drawings in which:

FIG. 1 illustrates a schematic representation of a process for preparing a colloid gel suitable for use as an implant.

FIG. 2 illustrates a schematic representation of a process of using shear thinning for preparing a colloid gel suitable for injection to form an implant.

FIGS. 3A-3D include micrographs from a scanning electron microscope (SEM) of colloidal gels, which micrographs illustrate similar porous microstructure and nanostructure for (FIGS. 3A and 3C) 1:1 and (FIGS. 3B and 3D) 7:3 (PLGA-PEMA:PLGA-PVAm) weight ratios in the dry state.

FIGS. 4A-4B include laser scanning confocal micrographs (LSCM) of colloidal gels (5% wt/vol), which illustrate that a 1:1 weight ratio contained nanoparticles organized into networks (FIG. 4A), but the 7:3 ratio did not exhibit similar long-range structure (FIG. 4B).

FIG. 5A includes a graph that illustrates that high viscosity and shear-thinning behavior can be observed in colloidal gels mixed at different ratios compared to pure nanoparticles for accelerating (solid symbols) and decelerating (open symbols) shear force.

FIG. 5B includes a graph that illustrates that increasing nanoparticle mass per volume of water systematically increased viscosity trends.

FIG. 5C includes a graph that illustrates that colloidal gels with a 1:1 mass ratio showed a steady decrease in viscosity for each cycle when no recovery time was allowed between shear cycles.

FIGS. 6A-6C illustrate that tissue scaffolds made from 20% wt/vol colloidal gels (1:1 mass ratio) can be formed into a variety of shapes, which FIG. 6C illustrating that the colloid gels have sufficient cohesiveness to be handled by a 20 gauge needle.

FIG. 6D includes a micrograph of human umbilical cord matrix stem cells cultured on colloidal gels demonstrated high viability (green; oblong cell shaped in gray scale) and minimal cell death (red; spots in grayscale).

FIGS. 7A-7B are photographs that compare a bone defect with and without treatment with the colloid gel tissue engineering scaffold.

FIG. 8A-8B include graphs that illustrate the encapsulation efficiency of drug loaded particles of a colloid gel and cumulative drug release from the colloid gel.

### DETAILED DESCRIPTION

Generally, the present invention includes three-dimensional tissue engineering scaffolds formed from biodegradable colloid gels that can be used as implants, prostheses, such as endoprostheses or exoprostheses, bandages, superficial tissue scaffolds, and topical tissue scaffolds. More particularly, the present invention relates to three-dimensional tissue engineering scaffolds that are prepared from particles, such as nanoparticles and/or microparticles, having opposite charges. A first group of particles can have a positive change and a second group of particles can have a negative charge such that a moldable tissue engineering scaffold can be prepared when the two different groups of particles with opposite charges are combined. The particles with opposite charges are attracted to each other so as to form a colloid gel that is configured for being moldable and implantable. The colloid gel is moldable before, during, or after implantation or application. The scaffold with the oppositely charged particles can form a scaffold that can be used in various tissue engineering applications and cells can grow on and within the scaffolds.

### I Colloidal Gel Scaffold

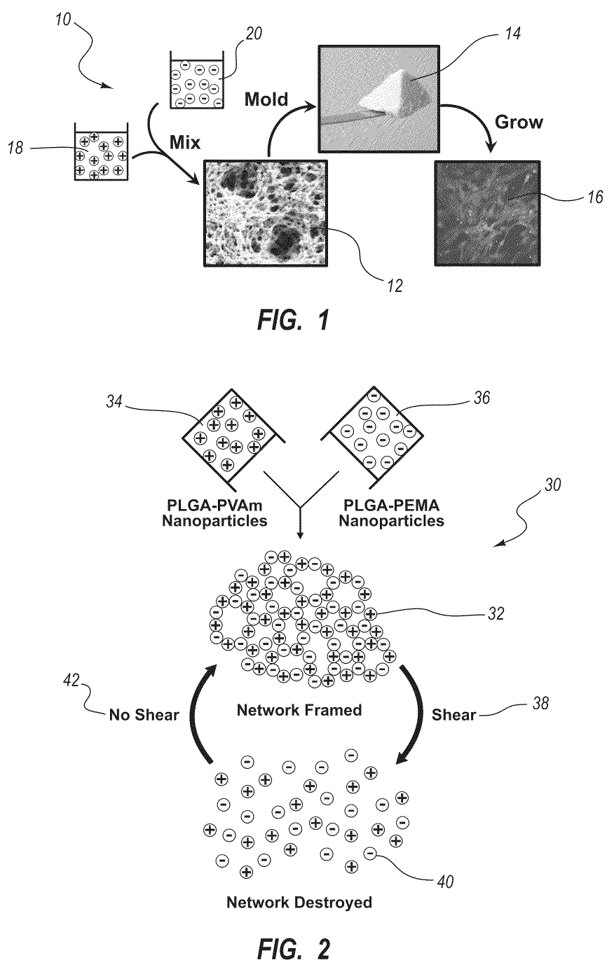

FIG. 1 provides a schematic representation of a process 10 for preparing a colloid gel 12 that can be formed into a molded into an implant 14 for use as a tissue engineering scaffold 16. As shown in FIG. 1, positive particles 18 and negative particles 20 can be combined to prepare a porous colloid gel 12. The colloid gel 12 can then be molded into an implant 14 that can have any of a variety of shapes. Often, the shapes will be in a form suitable for implantation. The implant 14 can then be implanted so that cells grow within the pores to provide use as a tissue engineering scaffold 16.

FIG. 2 provides another schematic representation of a process 30 for preparing a colloid gel 32 that can be injected in to a body and form an engineering scaffold in situ. As shown, positive particles 34 and negative particles 36 can be combined to prepare a porous colloid gel 32, which is a network framed with particles. The colloid gel 32 is substantially as described herein and includes a network of positive particles 34 that are associated with negative particles 36 in order to form a matrix with pores in the form of a colloid gel 32. The colloid gel 32 has a shear-thinning characteristic in that when a shear force 38 is applied to the colloid gel 32, such as from being injected from a syringe, passed through a tube, or being stirred, the positive particles 34 and negative particles 36 can become disassociated so as to form a paste 40 provide some fluidity to the colloid gel 32. Accordingly, the particle network can be destroyed to provide the fluidity. The fluidity can be similar to that of a paste such that the colloid gel 32 is moldable and can be shaped with a spatula or other utensil. When under no shear force 42, the positive particles 34 and negative particles 36 can again be combined to form the porous colloid gel 32. The colloid gel 32 can then set up into a structurally sound form when no shear is applied. Thus, the set up colloid gel 32 can be used as an implant and can be injected into a defect site within a body to provide a moldable and shapeable implant in situ.

The tissue engineering scaffold can be used for growing cells, and can include a first plurality of positively-charged biocompatible particles and a second plurality of negatively-charged biocompatible particles. The positive and negative particles can be linked together through ionic interactions or other interactions so as to form a three-dimensional matrix in the form of a colloid gel. Optionally, the matrix can include a plurality of pores defined by and disposed between the particles. The pores can be smaller than the particles or sized sufficient for receiving and growing living cells. For example, the pores can be the interstitial space between the particles or larger pores. Accordingly, the pores can be dimensioned to retain small molecules, macromolecules, cells, and the like. Also, the linked particles can have a surface area sufficient for growing cells within the plurality of pores and on the scaffold prepared from the particles.

The biocompatible particles can include first and second sets of particles. Generally, the first set of particles is positively charged and the second set of particles is negatively charged, or vice versa. Additionally, the first set of particles can have a first characteristic other than charge type. The second set of particles can have a second characteristic other than charge type that is different from the first characteristic. For example, the first and second characteristics can be independently selected from the group consisting of the following: composition; polymer; particle size; particle size distribution; zeta potential; charge density; type of bioactive agent; type of bioactive agent combination; bioactive agent concentration; amount of bioactive agent; rate of bioactive agent release; mechanical strength; flexibility; rigidity; color; radiotranslucency; radiopaqueness; or the like.

The oppositely-charged particles can be combined into a comingled spatial distribution such that positive particles are associated with negative particles in a repeating format to form a matrix. In some instances, a portion of the matrix can have more particles with one type of charge than the other, and the other type of particles can have a higher charge density. That is, more particles with a lower charge density can be combined with less particles with a higher charge density in order to form the colloid gel matrix.

In one embodiment, a colloid gel for use as a tissue engineering scaffold can be prepared by substituting only one of the particles with a polymer. This can include a plurality of positive charged polymers being combined with a plurality of negative charged particles, or a plurality of negative charged polymers being combined with a plurality of positive charged particles. The charged polymer can have various molecular weights; however, larger and/or longer polymers can be useful and more particle like. The polymer can be branched, crosslinked, or linear. The charged polymer can include a charge density similar to the particles. Also, the polymer can have a plurality of units that carry the charge. The polymer can be substituted for either a positive particle or a negative particle, and a particle of opposite charge of the polymer can be combined therewith in order to prepare a colloid gel having the properties described herein for use as a tissue engineering scaffold.

The colloid gel matrix can include bioactive agents contained in or disposed on a first set of particles or either charge. The bioactive agents can also be disposed in the interstitial spaces between the linked particles. The resulting scaffold can be configured to release the bioactive agents so as to create a desired concentration of bioactive agent. Optionally, a second set of particles can be substantially devoid of the bioactive agent, or can include a second bioactive agent. When the second bioactive agent is contained in or disposed on the second set of particles, the scaffold can be configured to release the second bioactive agent so as to create a desired concentration of the second bioactive agent that is the same or different from the first desired concentration of the first bioactive agent. The different bioactive agents can be in both positive and negative particles or in distinct particles. For example, the positive particles can include a first bioactive agent and the negative particles can include a second bioactive agent. Also, the positive particles can include more than one type of bioactive agent. Moreover, the same bioactive agent can be in both positive and negative particles. This allows for a diverse and complex configuration of particles so that desired release profiles of one or more bioactive agent can be obtained. Furthermore, particles with one type of agent can be preferentially disposed on one side of the colloid gel matrix with a different type of agent in a different side or portion of the matrix. The configuration of different particles with different bioactive agents can be achieved during the manufacturing process by locating one type of particle in one position within a mold and a different type of particle in a different position. Thus, a number of different types of particles can each have a bioactive agent to provide a plurality of different types of bioactive agents to the scaffold.

In one embodiment, the bioactive agent contained in a particle can be a growth factor for growing the cells. However, the particles can include any type of bioactive agent. Accordingly, the first characteristic of a first set of particles can be a first bioactive agent contained in or disposed on the particles, and the second characteristic of a second set of particles can be a second bioactive agent contained in or disposed on the particles. For example, the first bioactive agent can be an osteogenic factor and the second bioactive agent can be a chondrogenic factor.

In one embodiment, at least one of a first set or second set of particles can include a biodegradable polymer. For example, the particles can include a poly-lactide-co-glycolide or poly(lactic-co-glycolic acid) or PLGA or other similar polymer or copolymer.

In one embodiment, the scaffold can include a medium sufficient for growing cells disposed in the pores. The medium can be a cell culture media. Additionally, the medium can be a body fluid or tissue.

In one embodiment, the scaffold can include a plurality of cells attached to the plurality of particles and growing within the pores. The scaffold can include one cell type or a plurality of cell types. For example, the scaffold can include a first cell type associated with a first set of particles, and a second cell type associated with a second set of particles.

In one embodiment, the scaffold can include a third set of particles having a third characteristic other than charge that is the same or different from the first or second characteristics. The third set of particles can have a predetermined spatial location that is different from or the same as the spatial locations of the positive and negative particles with respect to the matrix. Also, the third set can be positive, negative, or neutral. When neutral, the particles can be entrapped within a matrix of positive/negative particles or can be chemically bound thereto.

In one embodiment, the scaffold can include a first end and an opposite second end. Accordingly, a first set of particles can have a first bioactive agent, and the first end can have a majority of particles of the first set. Correspondingly, a second set of particles can have a second bioactive agent that is different from the first bioactive agent, and the second end having a majority of particles of the second set.

### II. Method of Manufacture

Colloidal gels can be fabricated using oppositely-charged particles, such as nanoparticles or microparticles, which interact to form stable three-dimensional scaffolds. That is, the colloid gels can be molded and/or shaped into tissue engineering scaffolds for a variety of uses. The shaping can be done prior to implantation to form a stable structure or can be done during implantation so as to form the stable structure in situ. The scaffolds can be configured with a desired degree of malleability under shear and strong static cohesion so as to facilitate fabrication of shape-specific tissue scaffolds. Also, a charged polymer can be substituted for one of the charged particles during the manufacture process to produce a colloid gel having a charged particle and an oppositely charged polymer. As such, the descriptions herein can include one charged particle being substituted with a charged polymer.

The colloid gels can be prepared from biodegradable particles and/or biostable particles. As such, the particles can be polymeric, organic, inorganic, ceramic, minerals, combinations thereof, and the like. The colloid gels can include more than one type of particle, such as a biodegradable polymer and a mineral.

Colloidal gels can be prepared from oppositely-charged nanoparticles at high concentration exhibit pseudoplastic behavior that allows for the fabrication of shape-specific microscale materials. The cohesive strength of these materials depends upon interparticle interactions such as; electrostatic forces, van der Waals attraction, steric hindrance, and the like which may be leveraged to facilitate the synthesis of ceramic devices, sensors, or drug delivery systems.

A novel and cost-efficient method has been developed in order to create particle-based three-dimensional materials, which may be utilized in a variety of applications, such as tissue generation and/or regeneration. Moreover, with a suitable choice of biomaterial, it has been shown that the synthesis and encapsulation process is conducive to cell viability. Specifically, the technique can be used to create scaffolds that can be used in diverse areas of tissue engineering applications, including nerve tissue engineering, study of chemotaxis, angiogenesis, release of chemokines for modulating immune response, interfacial tissue engineering, and the like.

The process of making the three-dimensional tissue engineering scaffolds with oppositely charged particles successfully produces porous, well-connected matrices, which may be suitable for a variety of tissue engineering applications depending on the selection of suitable biomaterial(s). The process can be used to create porous, biocompatible and biodegradable scaffolds using particles made of, for example, poly(D,L-lactide-co-glycolide) (PLG), poly(D,L-lactic-co-glycolic acid) (PLGA). Additionally, porosity patterns can be created within a scaffold using particles of different sizes.

In one embodiment, the present invention can include a method of preparing tissue engineering scaffold for growing cells. Such a method can include the following: providing a first set of particles having a positive charge; providing a second set of particles having a negative charge; and combining the particles of the first set and second set together so as to form a three-dimensional matrix having a plurality of pores defined by and disposed between the particles. The plurality of particles can have a surface area sufficient for growing cells within the plurality of pores. The three-dimensional matrix can include the first set and second set of particles being comingled such that the positive particles are adjacent and ionically associated with the negative particles so as to form the matrix.

Scaffolds can be fabricated by flowing oppositely charged particle suspensions into a mold of pre-determined shape (to allow fabrication of shape-specific materials) with predefined flow profiles. The oppositely charged particles can be combined and mixed together so as to associate and form a continuous material. The process can utilize commercially available programmable syringe pumps (e.g., Motor-driven syringe pumps) to pump the oppositely charged particles into a mold. These types of pumps can now be used with oppositely charged particle compositions to create three-dimensional tissue engineering scaffolds with various characteristics. The method of manufacturing a tissue engineering scaffold with oppositely charged particles that associate into a matrix with a network of pores is a novel way to synthesize the products, with diversified area of application (e.g., useful for many applications, including tissue regeneration).

Also, freeform printing of the oppositely charged particle compositions can form colloidal gels that can be shaped by printing, molding, or cutting, to produce three-dimensional microperiodic networks exhibiting precise structure.

Additionally, the colloid gels can be molded and freeze dried to create more rigid structures or directly injected as in situ forming scaffolds. Application of porogens, such as sodium chloride, salts, oil, parafins, polymers, surfactants, and the like, to the scaffolds can create pores of various sizes so as to promote in-growth of cells and enhance interconnected pore 3-D structure. In addition, integration of controlled release strategies (e.g. growth factors) would be straightforward and would allow advanced combination strategies for tissue engineering coupled with growth factor delivery.

In one embodiment, the method of preparing a particle-based scaffold can include any one of the following: preparing a first liquid suspension of the first set of positive particles; preparing a second liquid suspension of the second set of negative particles; introducing the first liquid suspension into a mold; introducing the second liquid suspension into the mold before, during, and/or after introducing the first liquid suspension into the mold; molding the first and second set of particles into a mold with the positive charges associating with the negative charges so as to form a matrix.

In one embodiment, the first and second particles can be combined, and then introduced into a body of a subject to form the matrix. The matrix can then be shaped as needed or desired. For example, the first particle composition can be combined with the second particle composition, and the combined composition can be deposited into a desired location within the body of a subject. The desired location can be location in need of an implant, such as a bone defect or space, and the combined composition can be applied to the location and shaped. Thus, the composition can be pre-shaped prior to implantation or shaped after being deposited within a body of a subject.

In one embodiment, a bioactive agent is encapsulated within the particles. Encapsulation of bioactive agents into particles can be achieved during fabrication of the particles by including the bioactive agent with the composition that forms the particles. Any process of encapsulation can be used.

In one embodiment, the bioactive agent is disposed within the interstitial space between the particles. That is, the bioactive agent is mixed into the pores of the matrix.

In one embodiment, the present invention utilizes growth factor-encapsulated polymeric particles (or other biological agent-encapsulated particles) as constituents, which are long known to have capability for providing controlled, sustained release. For example, a colloid gel prosthesis can be prepared as a scaffold that is made from growth factor-loaded particles, which may serve as novel sustained delivery devices for applications in tissue engineering.

In one embodiment, the particles can include immobilized surface factors (e.g., RGD adhesion sequences). A distribution of particles having immobilized surface factors that produce a gradient of such factors can influence cell migration.

In one embodiment, a method for creating the particle-based scaffolds can be a performed by flowing two or more different types of distinct particles of opposite charges and differing in material, size, encapsulated bioactive signal, and/or tethered surface bioactive signal, and the like into a mold or other space at desired steady or varying rates. The shape of the final scaffold is determined by the shape of the mold, which can be any desired shape, for example a cylindrical “plug” shape.

An increase in the mechanical characteristics of the scaffolds can be achieved by particles with a bimodal distribution in the design of the scaffolds, which would provide additional connections between the particles and a closer packing.

In comparison to traditional particle preparation methods, the methods of the present invention provide the ability to prepare tissue engineering scaffolds from oppositely charged monodispersed particles, which may lead to improved systems to explore the effects of particle size and charge density on particle-based scaffolds. Scaffolds made of uniform particles are ideal to study the influence of particle size on the degradation patterns and rates within scaffolds. In addition, as observed in the case of colloidal gel tissue scaffolds, uniform particles can pack closely compared to randomly-sized particles, providing better control over the pore-sizes and porosity of the scaffold, and may considerably aid the mechanical integrity of the scaffolds. Moreover, local release of molecules from the particles in a bulk scaffold is related to individual particle size and polymer properties. Reproducibility and predictability associated with uniform particle-based scaffolds may make them suitable for a systematic study of physical and chemical effects in order to achieve control over local release of growth factor within such a scaffold. Various charge densities can also be used in a single scaffold.

In one embodiment, the particle-based scaffolds can be prepared from PLG or PLGA particles. However the particles can be prepared from substantially any polymer, such as biocompatible, bioerodable, and/or biodegradable polymers. Examples of such biocompatible polymeric materials can include a suitable hydrogel, hydrophilic polymer, hydrophobic polymer biodegradable polymers, bioabsorbable polymers, and monomers thereof. Examples of such polymers can include nylons, poly(alpha-hydroxy esters), polylactic acids, polylactides, poly-L-lactide, poly-DL-lactide, poly-L-lactide-co-DL-lactide, polyglycolic acids, polyglycolide, polylactic-co-glycolic acids, polyglycolide-co-lactide, polyglycolide-co-DL-lactide, polyglycolide-co-L-lactide, polyanhydrides, polyanhydride-co-imides, polyesters, polyorthoesters, polycaprolactones, polyesters, polyanhydrides, polyphosphazenes, poly(phosphoesters), polyester amides, polyester urethanes, polycarbonates, polytrimethylene carbonates, polyglycolide-co-trimethylene carbonates, poly(PBA-carbonates), polyfumarates, polypropylene fumarate, poly(p-dioxanone), polyhydroxyalkanoates, polyamino acids, poly-L-tyrosines, poly(beta-hydroxybutyrate), polyhydroxybutyrate-hydroxyvaleric acids, polyethylenes, polypropylenes, polyaliphatics, polyvinylalcohols, polyvinylacetates, hydrophobic/hydrophilic copolymers, alkylvinylalcohol copolymers, ethylenevinylalcohol copolymers (EVAL), propylenevinylalcohol copolymers, polyvinylpyrrolidone (PVP), poly(L-lysine), poly(lactic acid-co-lysine), poly(lactic acid-graft-lysine), polyanhydrides (such as poly(fatty acid dimer), poly(fumaric acid), poly(sebacic acid), poly(carboxyphenoxy propane), poly(carboxyphenoxy hexane), poly(anhydride-co-imides), poly(amides), poly(iminocarbonates), poly(urethanes), poly(organophasphazenes), poly(phosphates), poly(ethylene vinyl acetate) and other acyl substituted cellulose acetates and derivatives thereof, poly(amino acids), poly(acrylates), polyacetals, poly(cyanoacrylates), poly(styrenes), poly(vinyl chloride), poly(vinyl fluoride), poly(vinyl imidazole), chlorosulfonated polyolefins, polyethylene oxide, combinations thereof, polymers having monomers thereof, or the like. In certain preferred aspects, the nano-particles include hydroxypropyl cellulose (HPC), N-isopropylacrylamide (NIPA), polyethylene glycol, polyvinyl alcohol (PVA), polyethylenimine, chitosan, chitin, dextran sulfate, heparin, chondroitin sulfate, gelatin, etc. and their derivatives, co-polymers, and mixtures thereof. A non-limiting method for making nano-particles is described in U.S. Publication 2003/0138490, which is incorporated by reference.

The particles can be prepared from any mineral. For example, the mineral can be a mineral base, such as a mineral hydroxide, mineral oxide, and/or mineral carbonate. The mineral bases can include bases of potassium, magnesium, calcium, and combinations thereof, which can react with an acid to form a salt. Also, the mineral base can be an alkali or alkaline earth hydroxide, oxide, and/or carbonate. Preferably, the mineral is biocompatible. Examples of minerals that can also be used include mono, di, or trivalent cationic metals such as calcium, magnesium, manganese, iron, copper, zinc, potassium, cobalt, chromium, molybdenum, vanadium, sodium, phosphorus, selenium, lithium, rubidium, cesium, francium, and the like.

Furthermore, the particles can be formed from a ceramic material. In one aspect, the ceramic can be a biocompatible ceramic which optionally can be porous and of particle size described herein. Examples of suitable ceramic materials include hydroxylapatite, mullite, crystalline oxides, non-crystalline oxides, carbides, nitrides, silicides, borides, phosphides, sulfides, tellurides, selenides, aluminum oxide, silicon oxide, titanium oxide, zirconium oxide, alumina-zirconia, silicon carbide, titanium carbide, titanium boride, aluminum nitride, silicon nitride, ferrites, iron sulfide, and the like.

Moreover, the particles can include a radiopaque material to increase visibility during placement of the paste in situ that forms the scaffold. The radiopaque materials can be platinum, tungsten, silver, stainless steel, gold, tantalum, bismuth, barium sulfate, or a similar material.

The scaffolds can be prepared to contain and release substantially any therapeutic agent. Examples of some pharmaceutics agents that be useful in scaffolds for use in a body lumen, such as a blood vessel can include: anti-proliferative/antimitotic agents including natural products such as vinca alkaloids (i.e. vinblastine, vincristine, and vinorelbine), paclitaxel, epidipodophyllotoxins (i.e. etoposide, teniposide), antibiotics (dactinomycin (actinomycin D) daunorubicin, doxorubicin and idarubicin), anthracyclines, mitoxantrone, bleomycins, plicamycin (mithramycin) and mitomycin, enzymes (L-asparaginase which systemically metabolizes L-asparagine and deprives cells which do not have the capacity to synthesize their own asparagine); antiplatelet agents such as G(GP) II_{b}/IIIₐ inhibitors and vitronectin receptor antagonists; anti-proliferative/antimitotic alkylating agents such as nitrogen mustards (mechlorethamine, cyclophosphamide and analogs, melphalan, chlorambucil), ethylenimines and methylmelamines(hexamethylmelamine and thiotepa), alkyl sulfonates-busulfan, nirtosoureas (carmustine (BCNU) and analogs, streptozocin), trazenes-dacarbazinine (DTIC); anti-proliferative/antimitotic antimetabolites such as folic acid analogs (methotrexate), pyrimidine analogs (fluorouracil, floxuridine, and cytarabine), purine analogs and related inhibitors (mercaptopurine, thioguanine, pentostatin and 2-chlorodeoxyadenosine{cladribine}); platinum coordination complexes (cisplatin, carboplatin), procarbazine, hydroxyurea, mitotane, aminoglutethimide; hormones (i.e. estrogen); anti-coagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory; antisecretory (breveldin); anti-inflammatory: such as adrenocortical steroids (cortisol, cortisone, fludrocortisone, prednisone, prednisolone, 6α-methylprednisolone, triamcinolone, betamethasone, and dexamethasone), non-steroidal agents (salicylic acid derivatives e.g., aspirin; para-aminophenol derivatives i.e. acetaminophen; indole and indene acetic acids (indomethacin, sulindac, and etodalac), heteroaryl acetic acids (tolmetin, diclofenac, and ketorolac), arylpropionic acids (ibuprofen and derivatives), anthranilic acids (mefenamic acid, and meclofenamic acid), enolic acids (piroxicam, tenoxicam, phenylbutazone, and oxyphenthatrazone), nabumetone, gold compounds (auranofin, aurothioglucose, gold sodium thiomalate); immunosuppressives: (cyclosporine, tacrolimus (FK-506), sirolimus (rapamycin), everolimus, azathioprine, mycophenolate mofetil); angiogenic agents: vascular endothelial growth factor (VEGF), fibroblast growth factor (FGF); angiotensin receptor blockers; nitric oxide donors; antisense oligionucleotides and combinations thereof; cell cycle inhibitors, mTOR inhibitors, and growth factor receptor signal transduction kinase inhibitors; retenoids; cyclin/CDK inhibitors; HMG co-enzyme reductase inhibitors (statins); and protease inhibitors; β₂ agonists (e.g. salbutamol, terbutaline, clenbuterol, salmeterol, formoterol); steroids such glycocorticosteroids, preferably anti-inflammatory drugs (e.g. Ciclesonide, Mometasone, Flunisolide, Triamcinolone, Beclomethasone, Budesonide, Fluticasone); anticholinergic drugs (e.g. ipratropium, tiotropium, oxitropium); leukotriene antagonists (e.g. zafirlukast, montelukast, pranlukast); xantines (e.g. aminophylline, theobromine, theophylline); Mast cell stabilizers (e.g. cromoglicate, nedocromil); inhibitors of leukotriene synthesis (e.g. azelastina, oxatomide ketotifen); mucolytics (e.g. N-acetylcysteine, carbocysteine); antibiotics, (e.g. Aminoglycosides such as, amikacin, gentamicin, kanamycin, neomycin, netilmicin streptomycin, tobramycin; Carbacephem such as loracarbef, Carbapenems such as ertapenem, imipenem/cilastatin meropenem; Cephalosporins—first generation—such as cefadroxil, cefaxolin, cephalexin; Cephalosporins—second generation—such as cefaclor, cefamandole, defoxitin, cefproxil, cefuroxime; Cephalosporins—third generation—cefixime, cefdinir, ceftaxidime, defotaxime, cefpodoxime, ceftriaxone; Cephalosporins—fourth generation—such as maxipime; Glycopeptides such as vancomycin, teicoplanin; Macrolides such as azithromycin, clarithromycin, Dirithromycin, Erythromycin, troleandomycin; Monobactam such as aztreonam; Penicillins such as Amoxicillin, Ampicillin, Azlocillin, Carbenicillin, Cloxacillin, Dicloxacillin, Flucloxacillin, Mezlocillin, Nafcillin, Penicillin, Piperacillin, Ticarcillin; Polypeptides such as bacitracin, colistin, polymyxin B; Quinolones such as Ciprofloxacin, Enoxacin, Gatifloxacin, Levofloxacin, Lomefloxacin, Moxifloxacin, Norfloxacin, Ofloxacin, Trovafloxacin; Sulfonamides such as Mafenide, Prontosil, Sulfacetamide, Sulfamethizole, Sulfanilimide, Sulfasalazine, Sulfisoxazole, Trimethoprim, Trimethoprim-Sulfamethoxazole Co-trimoxazole (TMP-SMX); Tetracyclines such as Demeclocycline, Doxycycline, Minocycline, Oxytetracycline, Tetracycline; Others such as Chloramphenicol, Clindamycin, Ethambutol, Fosfomycin, Furazolidone, Isoniazid, Linezolid, Metronidazole, Nitrofurantoin, Pyrazinamide, Quinupristin/Dalfopristin, Rifampin, Spectinomycin); pain relievers in general such as analgesic and antiinflammatory drugs, including steroids (e.g. hydrocortisone, cortisone acetate, prednisone, prednisolone, methylpredniso lone, dexamethasone, betamethasone, triamcino lone, beclometasone, fludrocortisone acetate, deoxycorticosterone acetate, aldosterone); and non-steroid antiinflammatory drugs (e.g. Salicylates such as aspirin, amoxiprin, benorilate, coline magnesium salicylate, diflunisal, faislamine, methyl salicylate, salicyl salicylate); Arylalkanoic acids such as diclofenac, aceclofenac, acematicin, etodolac, indometacin, ketorolac, nabumetone, sulindac tolmetin; 2-Arylpropionic acids (profens) such as ibuprofen, carprofen, fenbufen, fenoprofen, flurbiprofen, ketoprofen, loxoprofen, naproxen, tiaprofenic acid; N-arylanthranilic acids (fenamic acids) such as mefenamic acid, meclofenamic acid, tolfenamic acid; Pyrazolidine derivatives such as phenylbutazone, azapropazone, metamizole, oxyphenbutazone; Oxicams such as piroxicam, meloxicam, tenoxicam; Coxib such as celecoxib, etoricoxib, lumiracoxib, parecoxib, rofecoxib (withdrawn from market), valdecoxib (withdrawn from market); Sulphonanilides such as nimesulide; others such as licofelone, omega-3 fatty acids; cardiovascular drugs such as glycosides (e.g. strophantin, digoxin, digitoxin, proscillaridine A); respiratory drugs; antiasthma agents; bronchodilators (adrenergics: albuterol, bitolterol, epinephrine, fenoterol, formoterol, isoetharine, isoproterenol, metaproterenol, pirbuterol, procaterol, salmeterol, terbutaline); anticancer agents (e.g. cyclophosphamide, doxorubicine, vincristine, methotrexate); alkaloids (i.e. ergot alkaloids) or triptans such as sumatriptan, rizatriptan, naratriptan, zolmitriptan, eletriptan and almotriptan, than can be used against migraine; drugs (i.e. sulfonylurea) used against diabetes and related dysfunctions (e.g. metformin, chlorpropamide, glibenclamide, glicliazide, glimepiride, tolazamide, acarbose, pioglitazone, nateglinide, sitagliptin); sedative and hypnotic drugs (e.g. Barbiturates such as secobarbital, pentobarbital, amobarbital; uncategorized sedatives such as eszopiclone, ramelteon, methaqualone, ethchlorvynol, chloral hydrate, meprobamate, glutethimide, methyprylon); psychic energizers; appetite inhibitors (e.g. amphetamine); antiarthritis drugs (NSAIDs); antimalaria drugs (e.g. quinine, quinidine, mefloquine, halofantrine, primaquine, cloroquine, amodiaquine); antiepileptic drugs and anticonvulsant drugs such as Barbiturates, (e.g. Barbexaclone, Metharbital, Methylphenobarbital, Phenobarbital, Primidone), Succinimides (e.g. Ethosuximide, Mesuximide, Phensuximide), Benzodiazepines, Carboxamides (e.g. Carbamazepine, Oxcarbazepine, Rufinamide) Fatty acid derivatives (e.g. Valpromide, Valnoctamide); Carboxilyc acids (e.g. Valproic acid, Tiagabine); Gaba analogs (e.g. Gabapentin, Pregabalin, Progabide, Vigabatrin); Topiramate, Ureas (e.g. Phenacemide, Pheneturide), Carbamates (e.g. emylcamate Felbamate, Meprobamate); Pyrrolidines (e.g. Levetiracetam Nefiracetam, Seletracetam); Sulfa drugs (e.g. Acetazolamide, Ethoxzolamide, Sultiame, Zonisamide) Beclamide; Paraldehyde, Potassium bromide; antithrombotic drugs such as Vitamin K antagonist (e.g. Acenocoumarol, Dicumarol, Phenprocoumon, Phenindione, Warfarin); Platelet aggregation inhibitors (e.g. antithrombin III, Bemiparin, Deltaparin, Danaparoid, Enoxaparin, Heparin, Nadroparin, Pamaparin, Reviparin, Tinzaparin); Other platelet aggregation inhibitors (e.g. Abciximab, Acetylsalicylic acid, Aloxiprin, Ditazole, Clopidogrel, Dipyridamole, Epoprostenol, Eptifibatide, Indobufen, Prasugrel, Ticlopidine, Tirofiban, Treprostinil, Trifusal); Enzymes (e.g. Alteplase, Ancrod, Anistreplase, Fibrinolysin, Streptokinase, Tenecteplase, Urokinase); Direct thrombin inhibitors (e.g. Argatroban, Bivalirudin, Lepirudin, Melagatran, Ximelagratan); other antithrombotics (e.g. Dabigatran, Defibrotide, Dermatan sulfate, Fondaparinux, Rivaroxaban); antihypertensive drugs such as Diuretics (e.g. Bumetanide, Furosemide, Torsemide, Chlortalidone, Hydroclorothiazide, Chlorothiazide, Indapamide, metolaxone, Amiloride, Triamterene); Antiadrenergics (e.g. atenolol, metoprolol, oxprenolol, pindolol, propranolol, doxazosin, prazosin, teraxosin, labetalol); Calcium channel blockers (e.g. Amlodipine, felodipine, dsradipine, nifedipine, nimodipine, diltiazem, verapamil); Ace inhibitors (e.g. captopril, enalapril, fosinopril, lisinopril, perindopril, quinapril, ramipril, benzapril); Angiotensin II receptor antagonists (e.g. candesartan, irbesartan, losartan, telmisartan, valsartan); Aldosterone antagonist such as spironolactone; centrally acting adrenergic drugs (e.g. clonidine, guanabenz, methyldopa); antiarrhythmic drug of Class I that interfere with the sodium channel (e.g. quinidine, procainamide, disodyramide, lidocaine, mexiletine, tocamide, phenyloin, encamide, flecamide, moricizine, propafenone), Class II that are beta blockers (e.g. esmolol, propranolol, metoprolol); Class III that affect potassium efflux (e.g. amiodarone, azimilide, bretylium, clorilium, dofetilide, tedisamil, ibutilide, sematilide, sotalol); Class IV that affect the AV node (e.g. verapamil, diltiazem); Class V unknown mechanisms (e.g. adenoide, digoxin); antioxidant drugs such as Vitamin A, vitamin C, vitamin E, Coenzime Q10, melanonin, carotenoid terpenoids, non carotenoid terpenoids, flavonoid polyphenolic; antidepressants (e.g. mirtazapine, trazodone); antipsychotic drugs (e.g. fluphenazine, haloperidol, thiotixene, trifluoroperazine, loxapine, perphenazine, clozapine, quetiapine, risperidone, olanzapine); anxyolitics (Benzodiazepines such as diazepam, clonazepam, alprazolam, temazepam, chlordiazepoxide, flunitrazepam, lorazepam, clorazepam; Imidaxopyridines such as zolpidem, alpidem; Pyrazolopyrimidines such as zaleplon); antiemetic drugs such as Serotonine receptor antagonists (dolasetron, granisetron, ondansetron), dopamine antagonists (domperidone, droperidol, haloperidol, chlorpromazine, promethazine, metoclopramide) antihystamines (cyclizine, diphenydramine, dimenhydrinate, meclizine, promethazine, hydroxyzine); antiinfectives; antihystamines (e.g. mepyramine, antazoline, diphenihydramine, carbinoxamine, doxylamine, clemastine, dimethydrinate, cyclizine, chlorcyclizine, hydroxyzine, meclizine, promethazine, cyprotheptadine, azatidine, ketotifen, acrivastina, loratadine, terfenadine, cetrizidinem, azelastine, levocabastine, olopatadine, levocetrizine, desloratadine, fexofenadine, cromoglicate nedocromil, thiperamide, impromidine); antifungus (e.g. Nystatin, amphotericin B., natamycin, rimocidin, filipin, pimaricin, miconazole, ketoconazole, clotrimazole, econazole, mebendazole, bifonazole, oxiconazole, sertaconazole, sulconazole, tiaconazole, fluconazole, itraconazole, posaconazole, voriconazole, terbinafine, amorolfine, butenafine, anidulafungin, caspofungin, flucytosine, griseofulvin, fluocinonide) and antiviral drugs such as Anti-herpesvirus agents (e.g. Aciclovir, Cidofovir, Docosanol, Famciclovir, Fomivirsen, Foscarnet, Ganciclovir, Idoxuridine, Penciclovir, Trifluridine, Tromantadine, Valaciclovir, Valganciclovir, Vidarabine); Anti-influenza agents (Amantadine, Oseltamivir, Peramivir, Rimantadine, Zanamivir); Antiretroviral drugs (abacavir, didanosine, emtricitabine, lamivudine, stavudine, tenofovir, zalcitabine, zidovudine, adeforvir, tenofovir, efavirenz, delavirdine, nevirapine, amprenavir, atazanavir, darunavir, fosamprenavir, indinavir, lopinavir, nelfinavir, ritonavir, saquinavir, tipranavir); other antiviral agents (Enfuvirtide, Fomivirsen, Imiquimod, Inosine, Interferon, Podophyllotoxin, Ribavirin, Viramidine); drugs against neurological dysfunctions such as Parkinson's disease (e.g. dopamine agonists, L-dopa, Carbidopa, benzerazide, bromocriptine, pergolide, pramipexole, ropinipole, apomorphine, lisuride); drugs for the treatment of alcoholism (e.g. antabuse, naltrexone, vivitrol), and other addiction forms; vasodilators for the treatment of erectile dysfunction (e.g. Sildenafil, vardenafil, tadalafil), muscle relaxants (e.g. benzodiazepines, methocarbamol, baclofen, carisoprodol, chlorzoxazone, cyclobenzaprine, dantrolene, metaxalone, orphenadrine, tizanidine); muscle contractors; opioids; stimulating drugs (e.g. amphetamine, cocaina, caffeine, nicotine); tranquillizers; antibiotics such as macrolides; aminoglycosides; fluoroquinolones and β-lactames; vaccines; cytokines; growth factors; hormones including birth-control drugs; sympathomimetic drugs (e.g. amphetamine, benzylpiperazine, cathinone, chlorphentermine, clobenzolex, cocaine, cyclopentamine, ephedrine, fenfluramine, methylone, methylphenidate, Pemoline, phendimetrazine, phentermine, phenylephrine, propylhexedrine, pseudoephedrine, sibutramine, symephrine); diuretics; lipid regulator agents; antiandrogen agents (e.g. bicalutamide, cyproterone, flutamide, nilutamide); antiparasitics; blood thinners (e.g. warfarin); neoplastic drugs; antineoplastic drugs (e.g. chlorambucil, chloromethine, cyclophosphamide, melphalan, carmustine, fotemustine, lomustine, carboplatin, busulfan, dacarbazine, procarbazine, thioTEPA, uramustine, mechloretamine, methotrexate, cladribine, clofarabine, fludarabine, mercaptopurine, fluorouracil, vinblastine, vincristine, daunorubicin, epirubicin, bleomycin, hydroxyurea, alemtuzumar, cetuximab, aminolevulinic acid, altretamine, amsacrine, anagrelide, pentostatin, tretinoin); hypoglicaemics; nutritive and integrator agents; growth integrators; antienteric drugs; vaccines; antibodies; diagnosis and radio-opaque agents; or mixtures of the above mentioned drugs (e.g. combinations for the treatment of asthma containing steroids and β-agonists); or any other biologically active agent such as nucleic acids, DNA, RNA, siRNA, polypeptides, antibodies, and the like. Growth factors and adhesion peptides can be useful for tissue development within a subject and can be included in the particles.

The particle-based scaffold can be prepared into substantially any shape by preparing a mold to have the desired shape or by shaping the colloid gel into a desired shape. For example, the particle-based scaffold can be prepared into the shapes of rods, plates, spheres, wrappings, patches, plugs, depots, sheets, cubes, blocks, bones, bone portions, cartilage, cartilage portions, implants, orthopedic implants, orthopedic screws, orthopedic rods, orthopedic plates, uneven shapes, random shapes, void space shapes, and the like. Also, the particle-based scaffolds can be prepared into shapes to help facilitate the transitions between tissues, such as between bone to tendon, bone to cartilage, tendon to muscle, dentin to enamel, skin layers, disparate layers, and the like. The particle-based scaffolds can also be shaped as bandages, plugs, or the like for wound healing. The shaping can be conducted within or outside of the body of a subject. Any utensil, such as various medical devices or sculpturing devices can be used to provide a shape to the colloid gel.

In one embodiment, the colloid gel scaffolds can be prepared in a manner so as to have pores. Since the material is a colloid gel with shear thinning, the pores can be formed with additives, poragens or cells can infiltrate the colloid gel so as to form pores. Cells and other substances can move into the colloid gel and push the particles around with force similar to shear thinning. When a cell or other substance penetrates into the colloid gel, a temporary or permanent pore may form. That is, the pathway formed by the cell can remain open, or other forces can close the pathway.

In one embodiment, the colloid gel scaffolds can be prepared to have a mean particle size of the particles used to prepare the scaffolds can have a wide range of sizes. The particles can be nanoparticles through microparticles, and the scaffolds can include both nanoparticles and microparticles. The nanoparticles can range between about less than 1 nm to about greater than 1 um (e.g., um is a micron), more preferably about 10 nm to about 500 nm, and most preferably from about 100 nm to about 250 nm. An example of particle size is about 180 nm to about 220 nm. The microparticles can range between about less than 1 um to about greater than 1 mm, more preferably about 10 um to about 500 um, and most preferably from about 100 um to about 250 um. An example of particle size is about 180 um to about 220 um.

The use of smaller particles can provide increased surface area, and thereby there is a lot more contact between particles. The smaller particles can create a material that is much more cohesive than expected, and the cohesive material behaves like a paste. Such pastes are useful for in situ injection of the colloid gel to form a scaffold during the injection. The paste can be used to fill bone defects or cartilage defects, and also can be used by it to apply to wounds as a filler.

In one embodiment, the colloid gel scaffolds can be prepared to have an average moduli of elasticity that can have a range between about 6 kPa to about 40 MPa, more preferably about 200 kPa to about 8 MPa, and most preferably from about 1 MPa to about 4 MPa. Examples of elasticity can be about 4.2 MPa to about 6.0 MPa or about 5 MPa to about 12 MPa. Once dried or cured, the scaffold can be much more rigid.

The colloid gels with oppositely charged nanoparticles provide a new material that has both biocompatibility and the ability to controllably release drugs or therapeutics. The flowability of the paste provides a mechanical property that is desirable, and the cohesiveness of the gel provides a shapeable, stable structure.

The paste format of the oppositely charged nanoparticles colloid gel allows for shear-thinning upon extrusion, and the paste can flow and then set up to be shape stable. For example, in a bone application it is desirable for the paste to be injectable so that it flows and sets up once it is in the defect site.

In one embodiment, the particle-based scaffold can be prepared with particles that include a core and one or more shells. Particles with core/shell configurations can be prepared by standard techniques. The core/shell configuration can allow for customized bioactive agent release profiles. For example, the shell can be configured to have one release rate and the core can have a second release rate. Also, the core can have a different bioactive agent compared to the shell. When multiple shells are used, the different shells can have different release rates and/or different bioactive agents.

### III. Methods of Use

The oppositely charged particles that form colloid gels for use as moldable scaffolds can advance tissue engineering. The colloid gels can be molded into shapes or configured into injectable compositions that can form tissue scaffolds in situ. The colloid gels are prepared in a way that provides control of material plasticity and recoverability by using to proven biodegradable materials. PLGA-based colloidal gels described herein can provide desirable properties for molding tissue scaffolds and demonstrated negligible toxicity to cells, such as HUCMSCs.

The colloid gels can be prepared into a prosthesis for internal or external use. The colloid gel can be implanted so as to be an endoprosthesis. Also, the colloid gel can be applied to a wound so as to be an exoprosthesis or bandage. The colloid gel can be used in a paste format and molded in situ, or the colloid gel can be hardened or cured into a more rigid and pre-shaped format and then implanted.

In one embodiment, the present invention can include a method of generating or regenerating tissue in an animal, such as a human. The method can include providing a prosthesis (e.g., endo or exo) for growing cells. An endoprosthesis can be deposited within a body, and an exoprosthesis can be deposited into a wound open to the surface. In both instances, the prosthesis can be used as a tissue engineering scaffold for growing cells. The colloid gel prosthesis can have a plurality of biocompatible positive and negative particles linked together so as to form a three-dimensional matrix having a plurality of pores defined by and disposed between the particles. Accordingly, the colloid gel prosthesis can include a particle-based scaffold. The plurality of positive and negative particles can have a surface area sufficient for growing cells within the plurality of pores. However, the positive particles may be more attractive to negatively charged cell membranes. The biocompatible particles can be characterized as described herein. Additionally, the method of generating or regenerating tissue can include implanting the prosthesis in the animal or placing the prosthesis into a wound such that cells grow on the particles and within the pores. This process can be used to grow specific types of cells for growth of tissue, bone, cartilage, or the like.

In one embodiment, the method of generating or regenerating tissue can include any one of the following: introducing a cell culture media into the pores of the matrix; introducing cells into the pores of the matrix; and/or culturing the cells such that the cells attach to the particles and grow within the pores. The cells can also grow in the outside of the matrix.

The colloid gel can be prepared as a paste for application to a wound or it can be prepared into a shaped bandage for application to the wound. The paste format allows for the colloid gel to form a tissue engineering scaffold that conforms to the shape of the wound so as to enhance wound healing. A bandage shape format can be used to superficial wounds and applied like a bandage that provides a scaffold for tissue growth.

The three-dimensional particle scaffolds can be used for the following: osteochondral defect repair (in the presence of growth factors with or without cells) and tissue engineering; axonal regeneration; study of chemotaxis in three-dimensions; directed angiogenesis; regeneration of other interfacial tissues such as muscle-bone, skin layers; control of release of inflammatory and/or immune system modulators in regenerative medicine applications; any application requiring a biocompatible, biodegradable material with control over material composition, bioactive signal release, and porosity; nerve regeneration; craniofacial and orthopedic applications; and the like.

In one embodiment, the particle-based scaffold can be used as an integrated osteochondral plug. Orthopedic surgeons can implant such a plug in a minimally invasive manner (arthroscope), with or without marrow or umbilical cord cells, to accelerate healing and allow osteoarthritis and impact-injury patients to return to load-bearing activities sooner. Conventional biodegradable plugs currently used have no bioactive signals to accelerate regeneration and do not account for the contrasting mechanical demands of the cartilage and underlying bone. More importantly, the particle-based scaffold technology is not limited to osteochondral applications, and can be used in any application where a gradient or integrated interface is desired, such as nerve regeneration, the ligament/bone interface, and the like.

In one embodiment, the present invention may be used in connection with a diverse type of eukaryotic host cells from a diverse set of species of the plant and animal kingdoms. Preferably, the host cells are from mammalian species including cells from humans, other primates, horses, pigs, and mice. For example, cells can be stem cells of any kind (e.g., umbilical cord or placenta derived, dental pulp derived, marrow-derived, adipose derived, induced stem cells, or cells of embryonic or amniotic origin), PER.C6 cells, HT-29 cells, LNCaP-FGC cells A549 cells, MDA-MB453 cells, HepG2 cells, THP-1 cells, miMCD-3 cells, HEK 293 cells, HeLaS3 cells, MCF7 cells, Cos-7 cells, CHO cells and CHO derivatives, CHO-K1 cells, BxPC-3 cells, DU145 cells, Jurkat cells, PC-3 cells, Capan-1 cells, HuVEC cells, HuASMC cells, HKB-11 human differentiated stem cells such as osteoblasts and adipocytes from hMSC; human adherent cells such as SH-SY5Y, IMR32, LAN5, HeLa, MCF10A, 293T, and SK-BR3; primary cells such as HUVEC, HUASMC, and hMSC; and other species such as 3T3 NIH, 3T3 L1, ES-D3, C2C12, H9c2 and the like. Additionally, any species of plant may be used.

### EXPERIMENTAL

1.

Oppositely charged PLGA nanoparticles were prepared by a solvent diffusion method. Briefly, 100 mg of PLGA was dissolved in 10.0 mL acetone and then the solution was added into 0.05% PVAm or PEMA (150 mL) through a syringe pump (20 mL/h) under stirring at 200 rpm overnight to evaporate acetone. Nanoparticles were collected by centrifugation (16,000 rpm, 20 min). The nanoparticles were washed using deionized water three times to remove excess surfactant. A fine powder of charged nanoparticles was obtained by lyophilization for ˜2 days.

PLGA dissolved in acetone was titrated into a water phase containing polyvinylamine (PVAm) or poly(ethylene-co-maleic acid) (PEMA) resulting in the precipitation of PLGA nanoparticles coated with the respective polyelectrolyte. The sizes and zeta potentials of the different PLGA nanoparticles were determined using a ZetaPALS dynamic light scattering system (Brookhaven, ZetaPALS). SEM was performed using a LEO 1550 field emission scanning electron microscope at an accelerating voltage of 5 kV. Laser scanning confocal microscopy was performed on an Olympus/Intelligent Innovations Spinning Disk Confocal Microscope with epifluorescence attachment.

The particle size of PLGA-PVAm nanoparticles was slightly smaller than that of PLGA-PEMA nanoparticles and the absolute value of the particle zeta potential of PLGA-PVAm nanoparticles was significantly larger than that of PLGA-PEMA nanoparticles. The polydispersity and zeta potential for the nanoparticles is shown in Table 1. These differences influenced gel properties since zeta potential and particle size are two critical factors influencing the properties of colloidal gel systems.

TABLE 1
PLGA Nanoparticle Properties

| | PLGA- | |
|---|---|---|
| | PEMA | PLGA-PVAm |
| Size (nm) | 181 ± 15 | 144 ± 12 |
| Polydispersity | 0.116 | 0.095 |
| Zeta potential (mV) | −20.1 ± 1.0 | +32.2 ± 1.3 |

2.

Colloidal gels exhibiting different degrees of cohesiveness were formed by mixing different ratios of positively and negatively charged PLGA nanoparticles and by controlling the total concentration of particles in suspension. The oppositely charged PLGA nanoparticles were combined to create a cohesive colloidal gel. The colloid self-assembled through electrostatic force resulting in a stable 3-D network that was easily molded to the desired shape. The colloidal gel demonstrated shear-thinning behavior due to the disruption of interparticle interactions as the applied shear force was increased. Once the external force was removed, the strong cohesive property of the colloidal gel was recovered. This reversibility makes the gel an excellent material for molding, extrusion, or injection of tissue scaffolds.

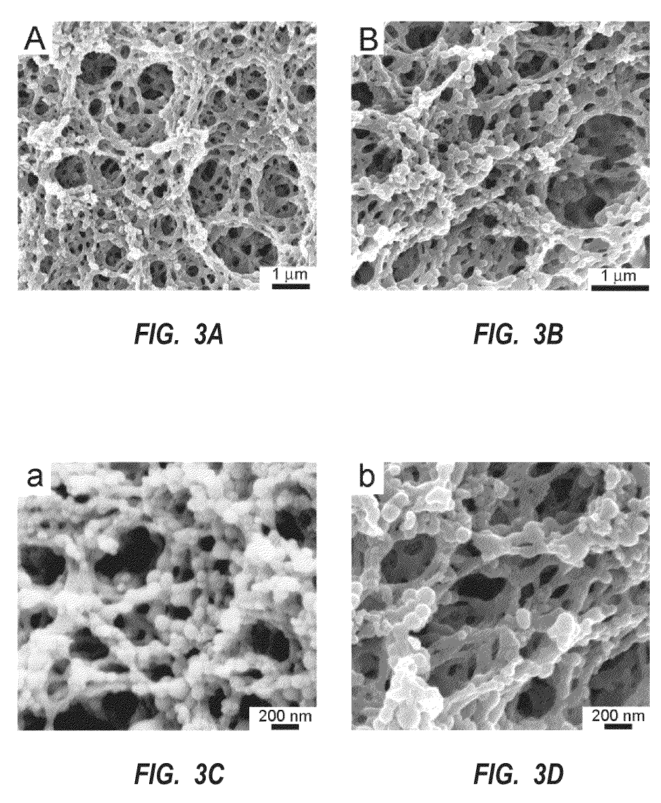

For initial studies, cationic or anionic nanoparticles were suspended in deionized water at 20% (w/w). Scanning electron micrographs of dried colloidal networks revealed little difference in the structure of dried gels containing different mass ratios of nanoparticles (FIGS. 3A-3D). When dried, each mass ratio (3:7, 1:1, and 7:3; PLGA-PEMA:PLGA-PVAm) exhibited a loosely organized, porous structure. Nanoparticles were linked together into micrometer-scale, ring-like structures, which interconnected to form the bulk porous structure observed. Domains of more tightly packed nanoparticle agglomerates were also evident suggesting that the cohesive nature of these colloidal gels results from an equilibrium of nanoparticle attraction (tight agglomerates) and repulsion (pores).

FIG. 3 is an SEM observation of colloidal gels revealed similar porous microstructure and nanostructure for (FIGS. 3A and 3C) 1:1 and (FIGS. 3B and 3D) 7:3 (PLGA-PEMA:PLGA-PVAm) weight ratios in the dry state.

3.

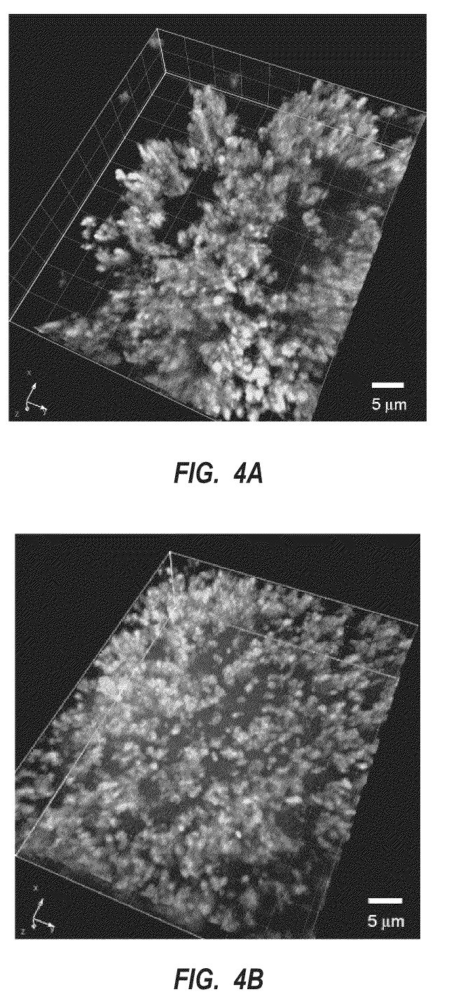

Laser scanning confocal microscopy (LSCM) was used to probe the structure of colloidal gels in solution. For this study, PLGA-PEMA nanoparticles were dyed with fluorescein (green) and PLGA-PVAm nanoparticles were dyed using rhodamine B (red). Colloidal gels were diluted by deionized water to 5% (w/w) for LSCM studies since high concentrations encumbered image acquisition. In FIGS. 4A-4B, laser scanning confocal micrographs (LSCM) of more dilute colloidal gels (5% wt/vol) revealed that (C) 1:1 weight ratio contained nanoparticles organized into networks, but (D) the 7:3 ratio did not exhibit similar long-range structure [PLGA-PEMA nanoparticles (green): PLGA-PVAm nanoparticles (red)]. 3-D projections of colloidal gels formed from mass ratios of 1:1 revealed long-range structure in the form of rings or bridges that were interconnected by more tightly agglomerated particles (FIG. 4A). 7:3 mass ratios appeared more homogeneous with discrete agglomerates of nanoparticles evident, but a lesser degree of long-range structure (FIG. 4B). These structures in situ supported the evidence of micro- and nanostructure of dried colloidal gels observed by SEM. 3-D LSCM composite images for 3:7 mass ratios were not attainable because of high particle mobility, which lead to image smearing during acquisition.

The 3:7 and 7:3 mass ratios of nanoparticles may behave similarly; however, colloidal gels composed of excess positively charged particles (3:7 mass ratio) exhibited more fluidity. LSCM video clips demonstrated the confined mobility of nanoparticles and fewer agglomerates compared to the 1:1 and 7:3 mass ratios (see supplementary video). In contrast, nanoparticles in colloidal gels comprising 1:1 and 7:3 mass ratios were essentially motionless. The larger zeta potential of positively charged nanoparticles resulted in a more equal overall charge balance when negatively charged particles were in excess, thus, providing a probable explaination for the stronger cohesion observed in the 7:3 mass ratio compared to the 3:7 mass ratio.

4.

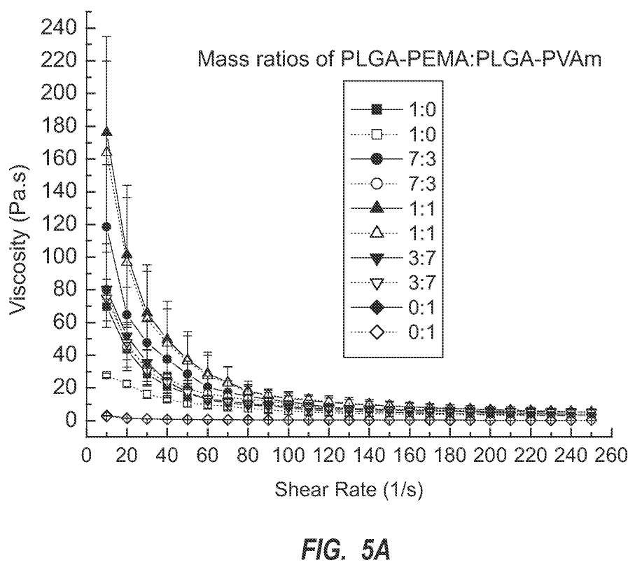

Rheological studies were employed to further probe the differences in plasticity of colloidal gels (FIGS. 5A-5C). Rheological experiments were performed by a controlled stress rheometer (AR2000, TA Instrument Ltd.). Flat steel plates (20 mm diameter) were used and the 500 μm gap was filled with colloidal gel. A solvent trap was used to prevent evaporation of water. The viscoelastic properties of the sample were determined at 20° C. by forward-and-backward stress sweep experiments. The viscosity (η) was monitored while the stress was increased and then decreased (frequency=1 Hz) in triplicate with 10 minutes between cycles. The gel recoverability was assessed using no time break between cycles.

Equal mass ratios of nanoparticles yielded the highest viscosity gel. As expected, mass ratios containing more negatively charged particles (7:3) exhibited higher viscosity than the inverse mass ratio. Pure nanoparticle suspensions exhibited minimal shear-thinning behavior. Viscosity was enhanced and shear-thinning more pronounced as the concentration of nanoparticles increased (FIG. 5B). Consecutive acceleration/deceleration cycles of the shear force revealed that these colloidal gels do not rapidly recover. Delaying shear cycles for more than one hour, however, enhanced the recovery of gel viscosity (FIG. 5C).

FIG. 5A shows that high viscosity and shear-thinning behavior were observed in colloidal gels mixed at different ratios compared to pure nanoparticles for accelerating (solid symbols) and decelerating (open symbols) shear force. FIG. 5B shows that increasing nanoparticle mass per volume of water systematically increased viscosity trends. FIG. 5C shows that colloidal gels with a 1:1 mass ratio showed a steady decrease in viscosity for each cycle when no recovery time was allowed between shear cycles.

5.

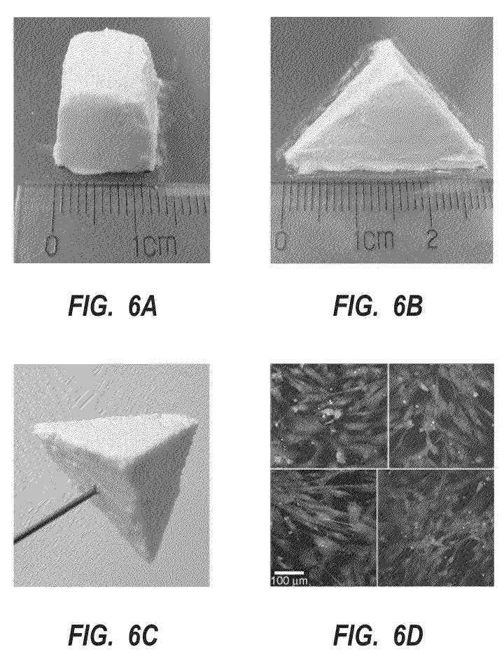

The pseudoplastic behavior of colloidal gels was leveraged to construct differently shaped tissue scaffolds (FIGS. 6A-6D). Molded scaffolds exhibited stable structure and shape retention when handled (FIG. 6C). The compatibility of colloidal gels with human umbilical cord matrix stem cells (HUCMSCs) was also assessed. For this study, colloidal gels were deposited and shaped in well plates.

FIGS. 6A and 6B show different shapes of tissue scaffolds made from 20% wt/vol colloidal gels (1:1 mass ratio). FIG. 6C shows that the colloid gels have sufficient cohesiveness to be handled by a 20 gauge needle without losing or changing shape.

6.

The colloid gels were studied for cell compatibility. Human umbilical cord matrix stem cells (HUCMSCs) were harvested and cultured until passage 1 as described previously described and then frozen in media consisting of 80% fetal bovine serum (FBS) and 20% dimethyl sulfoxide until use. Cells were thawed and expended to passage 4 for cell seeding at culture medium including low glucose Dulbecco's Modified Eagle's Medium, 20% FBS, and penicillin streptomycin (PS). HUCMSCs were seeded onto colloidal gels at a density of 1×10⁶ cells/mL. The colloidal gel was sterilized under UV light for 10 min. Cells were deposited on colloidal gels in the individual wells of a 24-well untreated plate, then 1 mL of defined medium was added into wells. ^{[27]} Cells were cultured in monolayer on the gel surface for 2 wks, with half of the media changed every other day. Subsequently, the scaffolds were stained with LIVE/DEAD reagent (dye concentration 2 mM calcein AM, 4 mM ethidium homodimer-1; Molecular Probes) and incubated for 45 min, before being subjected to fluorescence microscopy (Olympus/Intelligent Innovations Spinning Disk Confocal Microscope).

The scaffolds maintained integrity when culture media was introduced. HUCMSCs seeded onto the surface of the scaffolds were highly viable (green fluorescence), exhibiting minimal cell death (red fluorescence), which suggested that these colloidal gels were non-toxic to HUCMSCs (FIG. 6D). In addition, cell morphology was indicative of substantial cell adhesion to the scaffold.

FIG. 6D shows that human umbilical cord matrix stem cells cultured on colloidal gels demonstrated high viability (green; oblong in grayscale) and minimal cell death (red; spots in grayscale).

7.

In colloidal gel systems, the volume fraction (φ) and movement frequency (ω) of solid particles determines the viscosity of the system as described by:

η(φ, ω)=η₁(φ)+η₂(ω) (1)

The variable η is the viscosity of the colloidal system and is ascribed two parts: η₁ designated as the contribution of volume fraction of solid nanoparticles (increasing viscosity with higher fraction of solids, see FIG. 5B) and η₂ designated as the contribution of particle movement frequency as determined by interparticle interactions (e.g. electrostatic force, van der Waals attraction, steric repulsion). In cohesive colloidal gels, the movement frequency describes how easily a particle can escape from energy barriers associated with neighbor particles. Under static conditions, φ may strongly dictate the viscosity and structure of colloidal assemblies leading to a stable structure exhibiting high viscosity at equilibrium. If the particle-particle equilibrium is disrupted by an external force, the requisite activation energy for nanoparticle escape from the colloidal structure decreases simultaneously, thus, propagating a tendency towards viscosity reduction (shear-thinning) as the external force is increased. The composite balance of these attractive and repulsive forces under static conditions also directs the formation of the porous structures observed (FIGS. 3A-3D).
8.

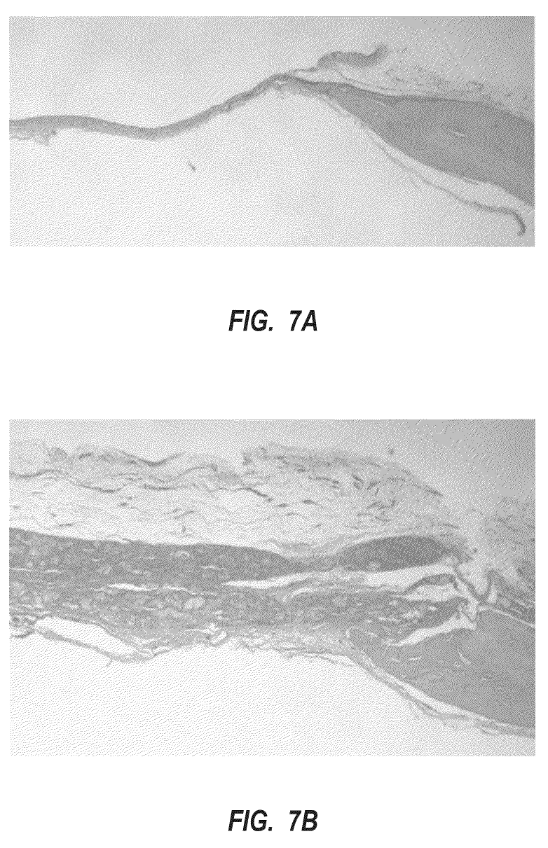

A colloidal gel made from PEMA- and PVAm-coated PLGA nanoparticles was injected into rat calvarial defects and studied for a period of 4 wks. Defects about 8 mm in diameter were created in the rat scull and, after 4 wks, the defect regions were harvested, decalcified, and stained (hematoxylin and eosin). Defect with the colloidal gel implant (with or without 10% dexamethasone) showed slightly more new bone at the defect periphery compared to the untreated defect (FIG. 7B). Untreated defects exhibited a thin layer of fibrous tissue and the defect had collapsed (FIG. 7A). The biomaterial effectively prevented the defects from collapse.

9.

Particles of PLGA-PVAm and PLGA-PEMA were prepared as drug-loaded particles. Briefly, 100 mg of PLGA was dissolved in 10.0 mL Dichloromethane as polymer stock solution; 10 mg Dexamethasone was dissolved in 1.0 mL Dichloromethane as drug stock solution; and the compositions were blended together at different ratios to get different drug loaded stock solution with drug concentration 5%, 10% and 20% (W/W). Then the drug loaded stock solution was added into 0.2% PVAm or PEMA (150 mL) through a syringe pump (60 mL/h) under homogenization at 15000 rpm to form drug loaded nanoparticles. After stirring at 200 rpm overnight to evaporate organic phase, drug loaded nanoparticles were collected by centrifugation (16,000 rpm, 20 min). The nanoparticles were washed using deionized water three times to remove excess surfactant. A fine powder of charged drug loaded nanoparticles was obtained by lyophilization for ˜2 days.

Lyophilized drug loaded nanoparticles (PLGA-PVAm or PLGA-PEMA) were dispersed in deionized water at 20% wt/vol. These dispersions were mixed in different proportions to obtain the different weight ratios drug loaded colloidal gel. Homogeneous colloid mixtures were prepared in a bath sonicator for 3 minutes and stored at 4° C. for 2 h to allow stabilization before use.

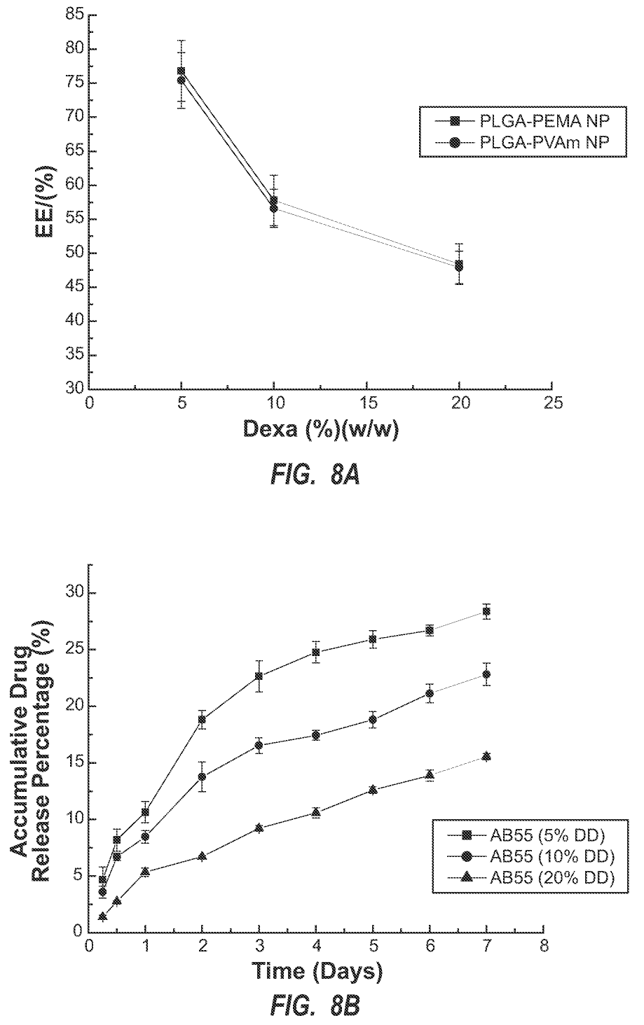

The colloid mixtures were than analyzed for the encapsulation efficiency of drug loading and drug release. FIG. 8A shows the encapsulation efficiency of both types of particles with dexamethason. FIG. 8B shows the release profile of different drug loadings.

10.

A PLGA-alginate/PLGA-Chitosan nanoparticle colloidal system was prepared. The particles were PLGA-alginate and PLGA-Chitosan. Briefly, chitosan was dissolved in 1% acetic acid solution and alginate was dissolved in distilled water. The surfactant concentration was 0.1%, 0.2%, 0.5% and 1% (w/w), respectively. 150 mg of PLGA was dissolved in 10.0 mL acetone and then the solution was added into Chitosan or Alginate (150 mL) solution through a syringe pump (20 mL/h) under stirring at 200 rpm overnight to evaporate acetone. Nanoparticles were collected by centrifugation (16,000 rpm, 20 min). The nanoparticles were washed using deionized water three times to remove excess surfactant. Then the particles (PLGA-Alginate or PLGA-Chitosan) were dispersed in deionized water at 20% wt/vol. These dispersions were mixed in different proportions to obtain the different weight ratios colloidal gels. Homogeneous colloid gels were prepared in a bath sonicator for 3 minutes and stored at 4° C. for 2 h to allow stabilization.

The sizes and zeta potentials of the different PLGA nanoparticles were determined using a ZetaPALS dynamic light scattering system (Brookhaven, ZetaPALS), which are shown in Table 2.

TABLE 2
1.5 g PLGA dissolved in 100 ml Acetone, 20 ml/h

| | 0.1% | | 0.2% | | 0.5% | | 1.0% | |
|---|---|---|---|---|---|---|---|---|
| Chitosan | 211.97 ± 9.8 | (nm) | 220.22 ± 11.7 | (nm) | 268.68 ± 9.8 | (nm) | 280.03 ± 11.7 | (nm) |
| | +7.61 ± 1.63 | (mV) | +14.96 ± 0.45 | (mV) | +19.69 ± 4.08 | (mV) | +21.03 ± 3.08 | (mV) |
| Alginate | 138.96 ± 2.4 | (nm) | 114.95 ± 3.2 | (nm) | 105.12 ± 1.6 | (nm) | 94.73 ± 1.8 | (nm) |
| | −27.58 ± 1.14 | (mV) | −26.17 ± 2.85 | (mV) | −26.45 ± 1.82 | (mV) | −23.21 ± 2.92 | (mV) |

11.

A particle and polymer colloid gel scaffold system was prepared and tested, and determined to form a colloid gel similar to the particle/particle system. Accordingly, a positive particle and negative polymer can be prepared into a colloid gel or a positive polymer and a negative particle can be prepared into a colloid gel. A colloid gel was prepared with PLGA-chitosan particles and alginate polymers. Briefly, chitosan was dissolved in 1% acetic acid solution with the concentration of 0.1%, 0.2%, 0.5% and 1% (w/w), respectively. Alginate was dissolved in water at 2% (W/W). 150 mg of PLGA was dissolved in 10.0 mL acetone and then the solution was added into Chitosan (150 mL) solution through a syringe pump (20 mL/h) under stirring at 200 rpm overnight to evaporate acetone. Nanoparticles were collected by centrifugation (16,000 rpm, 20 min). The nanoparticles were washed using deionized water three times to remove excess surfactant. Then the PLGA-Chitosan particles were dispersed in alginate solution in different proportions to obtain the different weight ratios composite gels. Homogeneous colloid gels were prepared in a bath sonicator for 3 minutes and stored at 4° C. for 2 h to allow stabilization. The PLGA-Chitosan nanoparticles were dispersed in same volume alginate solution to form composite gel. The polymer and particle colloid gel behaved similarly to particle and particle colloid gel system. This includes shape stability, shear thinning, and the like. For example, the polymer and particle colloid gel was placed into a vial and inverted, and the polymer retained the shape of the vial did not drop out of the vial.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope. All references recited herein are incorporated herein in their entirety by specific reference.

What is claimed is:

1. A biocompatible colloid gel comprising:
a plurality of positive charged biocompatible particles; and
a plurality of negative charged biocompatible particles associated with the plurality of positive charged particles so as to form a three-dimensional matrix having a plurality of pores defined by and disposed between the particles, said three-dimensional matrix having shear thinning under shear and structure stability in the absence of shear.

2. A colloid gel as in claim 1, wherein at least a portion of the plurality of positive charged particles and plurality of negatively charged particles are nanoparticles.

3. A colloid gel as in claim 1, wherein a majority of the plurality of positive charged particles and plurality of negatively charged particles are nanoparticles.

4. A colloid gel as in claim 1, wherein one of the plurality of positive charged particles or plurality of negative charged particles is a plurality of polymer molecules having the opposite charge of the other plurality of particles.

5. A colloid gel as in claim 1, wherein the colloid gel is disposed in a syringe.

6. A colloid gel as in claim 1, wherein the colloid gel is disposed within a subject.

7. A colloid gel as in claim 1, wherein the colloid gel is topically disposed in or on a wound of a subject.

8. A colloid gel as in claim 1, further comprising at least one bioactive agent disposed within the three-dimensional matrix.

9. A colloid gel as in claim 8, wherein the bioactive agent is disposed within at least one particle and/or within an interstitial space between the particles.

10. A colloid gel as in claim 1, further comprising cells disposed and growing within the pores.

11. A method for manufacturing a biocompatible colloid gel, the method comprising:
providing a plurality of positive charged biocompatible particles;
providing a plurality of negative charged biocompatible particles; and
combining the positive charged particles with the negative charged particles so as to form a three-dimensional matrix having a plurality of pores defined by and disposed between the positive and negative charged particles, said three-dimensional matrix having shear thinning under shear and structure stability in the absence of shear.

12. A method as in claim 10, further comprising preparing a majority of the plurality of positive charged particles and plurality of negatively charged particles as nanoparticles.

13. A method as in claim 12, wherein one of the plurality of positive charged particles or plurality of negative charged particles is a plurality of polymer molecules having the opposite charge of the other plurality of particles.

14. A method as in claim 10, further comprising introducing the colloid gel into a syringe.

15. A method as in claim 10, further comprising introducing the colloid gel into a subject as an implant.

16. A method as in claim 10, wherein the positive charged particles are adjacent and ionically associated with the negative charged particles so as to form the three-dimensional matrix and pores.

17. A method as in claim 1, further comprising introducing the colloid gel into or onto a wound of a subject.

18. A method as in claim 10, further comprising introducing at least one bioactive agent into the three-dimensional matrix.

19. A method as in claim 18, further comprising introducing the bioactive agent into at least one particle and/or an interstitial space between the particles.

20. A method as in claim 10, further comprising introducing cells into the pores.

21. A method of forming an implant in situ, the method comprising:
providing a colloid gel formed by combining positive charged particles with negative charged particles so as to form a three-dimensional matrix having a plurality of pores defined by and disposed between the positive and negative charged particles, said three-dimensional matrix having shear thinning under shear and structure stability in the absence of shear; and
injecting the colloid gel into a subject so as to form an implant.

22. A method as in claim 21, further comprising:
preparing a majority of the plurality of positive charged particles and plurality of negatively charged particles as nanoparticles; and
combining the positive charged particles and plurality of negatively charged particles to form the colloid gel.

23. A method as in claim 22, wherein one of the plurality of positive charged particles or plurality of negative charged particles is a plurality of polymer molecules having the opposite charge of the other plurality of particles.

24. A method as in claim 21, further comprising introducing the colloid gel into a syringe.

25. A method as in claim 21, further comprising shaping the colloid gel into a shape of the implant while within the subject.

26. A method as in claim 21, wherein the positive charged particles are adjacent and ionically associated with the negative charged particles so as to form the three-dimensional matrix and pores.

27. A method as in claim 21, further comprising introducing at least one bioactive agent into the three-dimensional matrix prior to the injecting.

28. A method as in claim 27, further comprising introducing the bioactive agent into at least one particle.

29. A method as in claim 27, further comprising introducing the bioactive agent into an interstitial space between the particles.

30. A biocompatible colloid gel for use in tissue engineering comprising:
a plurality of charged biocompatible particles having a first charge; and
a plurality of charged biocompatible polymers having a charge opposite of the first charge associated with the plurality of charged particles having the first charge so as to form a three-dimensional matrix, said three-dimensional matrix having shear thinning under shear and structure stability in the absence of shear.
